(19) United States
(12) Patent Application Publication
(10) Pub. No.: US 2025/0352128 A1
(43) Pub. Date: Nov. 20, 2025

(54) ROLLER DEVICE WITH DIFFERENTIAL/RATIOMETRIC ANALYTICS FOR CANCEROUS/NON-CANCEROUS LUMP DETECTION ON BREASTS USING SURFACE-ROLL AXIS PRESSURE

(71) Applicant: UNIVERSITY OF MARYLAND, BALTIMORE COUNTY, Baltimore, MD (US)

(72) Inventors: Govind Rao, Ellicott CIty, MD (US); Venkatesh Srinivasan, Baltimore, MD (US)

(21) Appl. No.: 19/205,403

(22) Filed: May 12, 2025

Related U.S. Application Data
(60) Provisional application No. 63/647,107, filed on May 14, 2024.

(51) Int. Cl. A61B 5/00; A61B 5/11

(52) U.S. Cl. CPC A61B 5/4312; A61B 5/11; A61B 5/7264; A61B 2562/0247; A61B 2562/046

(57) ABSTRACT

A device and a method for screening for the presence of lumps in breast tissue as well as the characterization of any discovered lumps including, but not limited to, location, size, shape, hardness, and movability.

### CROSS REFERENCE TO RELATED APPLICATIONS

This application claims priority to U.S. Provisional Patent Application No. 63/647,107 filed on May 14, 2024 in the name of Govind RAO, et al., entitled “Roller Device with Differential/Ratiometric Analytics for Cancerous/Non-Cancerous Lump Detection in Breasts Using Surface-Roll Axis Pressure,” which is hereby incorporated by reference herein in its entirety.

### FIELD

The present invention relates to a device and method for detecting the presence of lumps in breast tissue as well as evaluating lump morphology and movability to provide insight into lump malignancy. The device and method is dual purpose, being capable of a non-rolling use for screening for the presence of lumps and a rolling use for evaluating the lump's movability. Moreover, the device and the method enables differential/ratiometric evaluation for detecting lumps present in breast tissue regardless of breast tissue density.

### BACKGROUND

Breast cancer is the second most common cancer among women in the United States, with almost 1.3 million new cases of breast cancer reported between 2015-2019, leading to the death of 209,755 women. Two most important strategies have been laid out by the American Cancer Society to prevent deaths by breast cancer; early diagnosis and state-of-the-art treatment.

Currently, imaging techniques such as mammography and ultrasound are primarily used for early detection but face barriers such as limited access, high costs, and frequency constraints, hampering widespread screening efforts. Self and clinical breast examination (SBE/CBE) based screening involves manual palpation by self or healthcare professionals. However, manual palpation is subjective and has limitations in accuracy, risk of misdiagnosis, and an inability to provide longitudinal tumor progression data.

Currently, there is no affordable and accurate technology to conduct SBE/CBE for breast cancer detection without requiring high-end equipment. The current method is a primitive manual protocol with the use of finger pads to find lumps leading to a large number of false-positives and overdiagnosis due to subjective interpretation without reference or training.

Further, devices available for SBE/CBE suffer from gross inaccuracy and misdiagnosis and lack of longitudinal correlation as the woman has to rely on her memory about the conditions of her previous self-exam. Although there are devices aiding SBE/CBE, such as iBreastExam and infrared (IR) light-based monitors, they are either accurate but at a high cost or affordable and inaccurate. There are numerous IR light devices that are meant to help detect the lumps as opaque spots. However, this method is highly subjective and isn't applicable to all women owing to differences in muscle content and density variations. In fact, the Food and Drug Administration (FDA) has advised against the use of IR-based screening. The commercial device iBreastExam is accurate in detecting lumps, however it is expensive and does not evaluate movability of any detected lumps to score the malignancy.

Moreover, presently there are no available SBE/CBE devices able to detect triple-negative breast cancer (TNBC), which is cancer that is undetectable using three common tests; an estrogen test (ES), a progesterone test (PR) and a HER2 protein test. TNBC accounts for 10-15% of all breast cancers and spreads faster than other types of breast cancers. TNBC is more common among women of color and women younger than 40 years. Spatio-temporal breast analysis or biopsy are presently the only accurate ways to detect TNBC in time for treatment.

Given the high rate of incidence of breast cancer and unavailability of affordable self-examination technology, there remains a need in the art for a device that can aid with SBE/CBE as well as assist in the identification of the high-risk TNBC.

### SUMMARY

In some aspects, a system for detecting a lump in breast tissue is described, said system comprising:
a handheld device comprising a housing comprising a curved surface comprising an array of pressure sensors or electronic tactile sensors;
a processor configured to control the sensors and collect data therefrom; and
software configured to display and analyze the compiled data.

In some other aspects, a method for determining a presence, position, and movability of a lump in breast tissue is described, the method comprising:
using a system to collect non-rolling data to obtain a 3D map of breast tissue to determine if a lump is present in the breast tissue; and
if a lump is detected, using the system to collect rolling data in proximity of the lump to obtain a 3D map of movability of the lump to evaluate malignancy of the lump,
wherein the system comprises:
   a handheld device comprising a housing comprising a curved surface comprising an array of pressure sensors or electronic tactile sensors;
   a processor configured to control the sensors and collect data therefrom; and
   software configured to display and analyze the compiled data.

In still other aspects, a method of detecting breast cancer in a subject is described, the method comprising:
using a handheld device to collect non-rolling data;
using non-rolling data to obtain a 3D map of differential/ratiometric pressure data of the breast tissue to obtain a 3D map of breast tissue;
using AI/ML to detect the presence and position of a lump in the breast tissue;
using the handheld device to collect rolling data in proximity of the lump;
using the rolling data to obtain a 3D map of movability of the lump; and
using AI/ML to evaluate malignancy of the lump,
   wherein the handheld device comprises a housing comprising a curved surface comprising an array of pressure sensors or electronic tactile sensors.

Other aspects, features and embodiments of the invention will be more fully apparent from the ensuing disclosure and appended claims.

### BRIEF DESCRIPTION OF THE FIGURES

FIG. 1 is an illustration of an embodiment of the roller device with a curved surface sensor.

FIG. 2 is an illustration of an embodiment of the roller device with curved surface sensor.

FIG. 3 is an illustration of a perspective view of an embodiment of the roller device with curved surface sensor.

FIG. 4 is a schematic of varying radius and arc length of an embodiment of the curved surface sensor.

FIG. 5 is a schematic of varying radius and arc length of an embodiment of the curved surface sensor.

FIG. 6 illustrates an embodiment of the flow of the operation of the roller device, screening protocols, and analytics modules.

FIG. 7 is a schematic demonstrating the use of a roller device comprising a curved surface sensor to detect a lump and evaluate its movability.

FIG. 8 is a 3D view of another embodiment of the roller device described herein, comprising a roller ball.

FIG. 9 is a 3D view of another embodiment of the roller device described herein, comprising a cylindrical roller.

FIG. 10 is a 3D line diagram of the cylindrical roller, e.g., of FIG. 9, illustrating the dual pressure sensing locations for differential and ratiometric analysis.

### DETAILED DESCRIPTION, AND PREFERRED EMBODIMENTS THEREOF

Although the claimed subject matter will be described in terms of certain embodiments, other embodiments, including embodiments that do not provide all of the benefits and features set forth herein, are within the scope of this disclosure as well. Various structural and parameter changes may be made without departing from the scope of this disclosure.

Unless otherwise defined, all technical and scientific terms used herein have the same meaning as commonly understood by one of ordinary skill in the art. In case of conflict, the present document, including definitions, will control. Preferred methods and materials are described below, although methods and materials similar or equivalent to those described herein can be used in practice or testing of the present disclosure. All publications, patent applications, patents, and other references mentioned herein are incorporated by reference in their entirety. The materials, methods, and examples disclosed herein are illustrative only and not intended to be limiting.

“About” and “approximately” are used to provide flexibility to a numerical range endpoint by providing that a given value may be “slightly above” or “slightly below” the endpoint without affecting the desired result, for example, +/−5%.

The phrase “in one embodiment” or “in some embodiments” as used herein does not necessarily refer to the same embodiment, though it may. Furthermore, the phrase “in another embodiment” as used herein does not necessarily refer to a different embodiment, although it may. Thus, as described below, various embodiments of the invention may be readily combined, without departing from the scope or spirit of the invention.

The terms “comprise(s),” “include(s),” “having,” “has,” “can,” “contain(s),” and variants thereof, as used herein, are intended to be open-ended transitional phrases, terms, or words that do not preclude the possibility of additional acts or structures. The singular forms “a,” “and” and “the” include plural references unless the context clearly dictates otherwise. The present disclosure also contemplates other embodiments “comprising,” “consisting of” and “consisting essentially of,” the embodiments or elements presented herein, whether explicitly set forth or not.

As defined herein, to “roll,” “rolled,” “rolling,” “rocked” or “rocking” corresponds to both translational and rotational motion of the object, wherein the object rotates along a rolling axis as it translates from point C to point D on the surface. It should be appreciated that to “roll” or “rolling” includes the substantial minimization or substantial elimination of “sliding.” As used herein, “sliding” means that the object with the curved surface experiences the same point of contact at two different time instances during translation along the surface from point C to point D.

As used herein, the “rolling device” may comprise a curved surface that rolls about an axis 360° or alternatively, a curved surface that can be rocked back and forth with both translational and rotational motion.

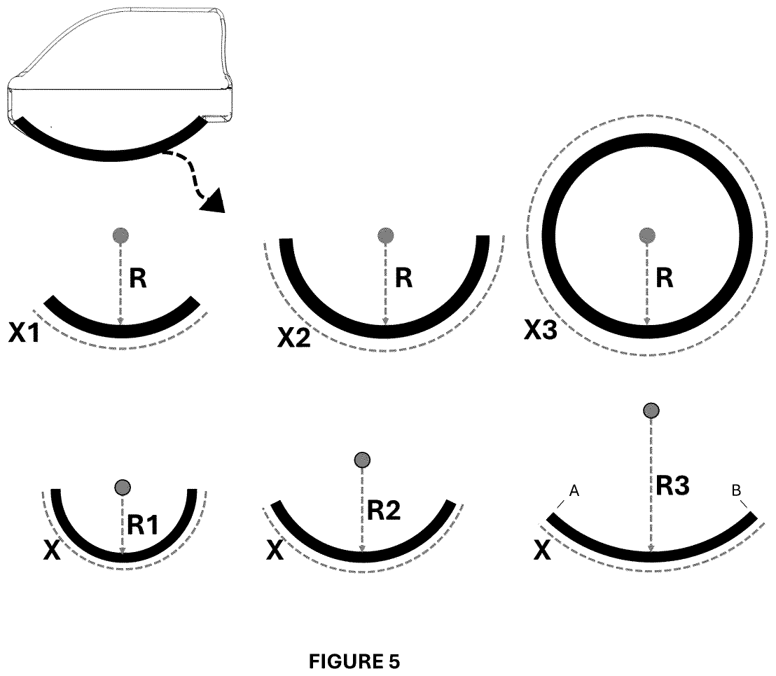

As defined herein, the “arc length” or “rolling distance” is the longest distance along the curved surface of the roller device from point A to point B in the array (see, e.g., FIGS. 2 and 5). It will be appreciated that the points A and B correspond to the outer sensors along the curved surface, wherein points A and B can correspond to a line of sensors, as illustrated in FIG. 2.

As used herein, a “lump” is a localized area of harder tissue of nodules within the soft breast tissue.

As used herein, a “subject” can be a human or other mammal prone to breast cancer, whether born male or female or hermaphroditic.

The various functions described herein may be implemented or supported by one or more computer programs, each formed from computer-readable program code and embodied in a computer-readable medium. The terms “application” and “program” refer to one or more computer programs, software components, sets of instructions, procedures, functions, objects, classes, instances, related data, or a portion thereof adapted for implementation in suitable computer readable program code. The phrase “computer readable program code” includes any type of computer code, including source code, object code, and executable code. The phrase “computer readable medium” includes any type of medium capable of being accessed by a computer, such as Read Only Memory (ROM), Random Access Memory (RAM), a hard disk drive, a Compact Disc (CD), a Digital Video Disc (DVD), or any other type of memory. A “non-transitory” computer-readable medium does not include a wired, wireless, optical, or other communication link that transmits transitory electrical or other signals. Non-transitory computer readable media include media that can permanently store data as well as media that can store data and later rewrite the data, such as rewritable optical disks or erasable memory devices.

The term “AI model” means a mathematical or statistical model that may be generated through artificial intelligence techniques such as machine learning and/or deep learning. For example, these techniques may involve inputting data into a neural network (e.g., an artificial neural network (ANN)) algorithm for training, so as to generate a model that can make predictions or decisions on new data without being explicitly programmed to do so. Different software tools (e.g., TensorFlow™, PyTorch™, Keras™) may be used to perform machine learning processes. Algorithms can identify statistical patterns that ANN recognizes as “local behavior” of the imaged materials which leads to simplified diagnosis and prognosis.

The present invention relates to a handheld diagnostic device and associated method for breast cancer screening through tactile sensing and pressure data analysis. More specifically, a device is described comprising an array of pressure or electronic tactile sensors mounted on a curved surface, configured to apply pressure to, or roll over, the breast to collect diagnostic data regarding the presence, position, and characteristics of potential lumps in breast tissue.

The device further enables classification of breast density by analyzing differential pressure responses between normal tissue and identified lumps. The device and method of using same collectively facilitate the detection of a lump, and if any lumps are found, at least one of (i) the lump position; (ii) the lump size and shape; (iii) the lump movability; and (iv) a classification of overall breast density, thereby enhancing early detection and evaluation of breast cancer.

Broadly, the roller device described herein employs a 3D visualization scheme to scan and map the breast tissue, complete with a time stamp, thus permitting the user to track changes and enable the obtainment of longitudinal analytics of changes in breast tissue over time, further assisting with the early detection of breast cancer. The roller device is a smart sensor that uses differential pressure analysis to characterize a breast and if a lump is detected, characterize the lump, using a single device. With the aid of artificial intelligence (AI) and machine learning (ML), the collected data can be stratified in real-time to differentiate malignant lumps from non-malignant ones.

In a first aspect, a handheld device is described, said device comprising a housing comprising a curved surface comprising an array of pressure sensors or electronic tactile sensors.

In a second aspect, a system is described, said system comprising:
a handheld device comprising a housing comprising a curved surface comprising an array of pressure sensors or electronic tactile sensors;
a processor configured to control the sensors and collect data therefrom; and
software configured to display and analyze the compiled data.

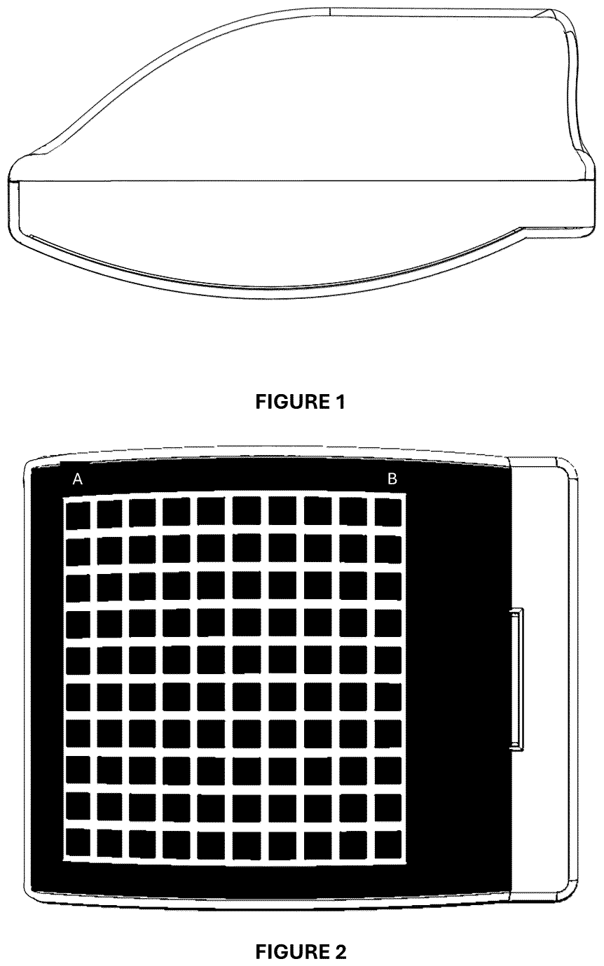

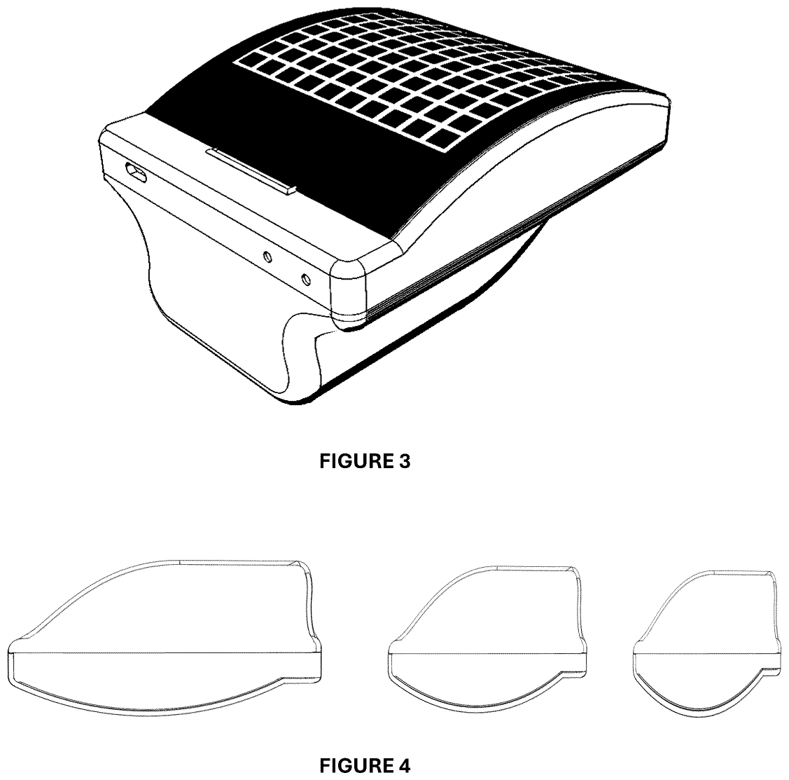

Referring to FIG. 1, an illustration of the side view of an embodiment of the handheld device is shown, complete with curved surface. Referring to FIG. 2, a surface of the device showing the curved surface embedded with an array of pressure sensors/tactile sensors is shown. The resolution of the array, or the number of individual sensors in a given area, can vary depending on the intended resolution and pressure ranges of the device. In some embodiments, the handheld device comprises hundreds of sensors in the array. In some other embodiments, the handheld device comprises thousands of sensors in the array. FIG. 3 illustrates a perspective view of an embodiment of the device showing the housing comprising the sensor array and a port, e.g., a USB-C, for charging and data transfer. It should be appreciated by the person skilled in the art that the array of pressure sensors/tactile sensors shown in FIGS. 2-3 are not intended to limit the arrangement and resolution of the array of the device described herein.

In some embodiments, the “curvature” of the curved surface can have a different radius and is defined as the distance between the surface of the sensors and the rolling axis of the device. For example, as illustrated in FIG. 4, three example devices with different curvature levels of the rolling sensor surface are shown. FIG. 5 shows possible variations in the device curved surface. The top row shows the variability of the arc length, i.e., rolling distances (X1, X2, X3) for a curved surface having same radius (R). The bottom row shows the variability of the radius of the curved surface (R1, R2, R3) while maintaining the same arc length or rolling distance (X).

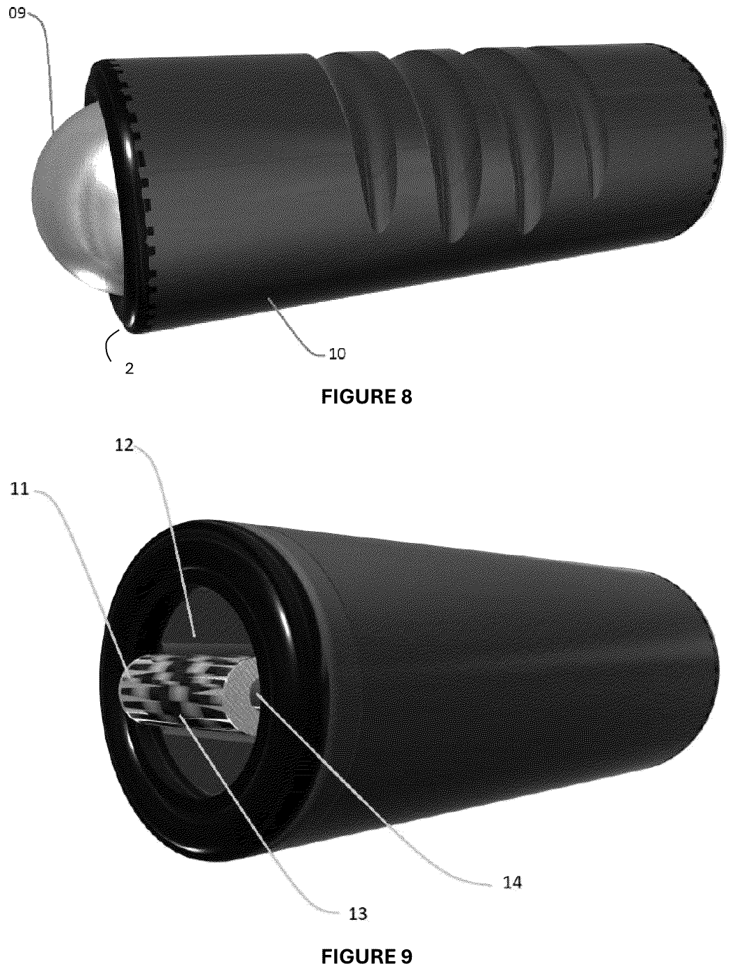

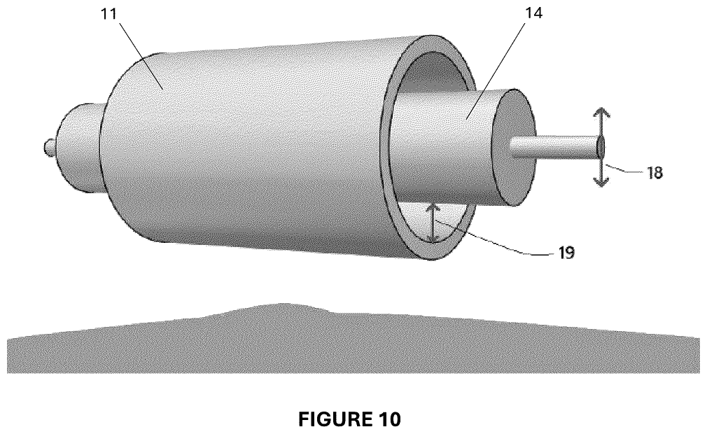

Additional embodiments of the handheld device are shown in FIGS. 8 and 9 herein. In FIG. 8, the handheld device has two ends: one having the roller ball having surface pressure sensors (9) and the other end optionally comprising transdermal sensors such as CO₂, O₂, humidity and/or temperature (not shown), in a housing (10), further comprising a roll axis pressure sensor (2). In FIG. 9, the handheld device also has two ends: one having the cylindrical roller (11) embedded with surface pressure sensors (13), wherein a second set of pressure sensors are housed at the roll axis holder (14), and the other end optionally comprising transdermal sensors such as CO₂, O₂, humidity and/or temperature (not shown), in a housing. Additional detail relating to the cylindrical roller of FIG. 9 can be seen in FIG. 10. In FIG. 10, the surface of the rolling cylinder (11) comprises the embedded surface pressure sensors (13) (not shown in FIG. 10). FIG. 10 more clearly illustrates the two layers of the rolling cylinder embodiment, wherein two pressures are monitored, a pressure sensor (18) for the axis holder (14) and a surface assembly sensor (19). In the embodiment of FIG. 10, the smart spatio-temporal mapping will aid in non-invasive breast mapping through pressure sensor rollers to locate a mass or lump as well as map the location, determine the size/shape, density, nodularity, durity and movability of mass/lump. This permits the user to stratify malignant/non-malignant lumps using compact metal/plastic rollers enabled with piezoelectric sensors and auto-tracking of roller movement and obstruction analysis.

In some embodiments, the curved surface of the device is manually operated to roll over the breast, wherein the rolling motion is defined as a process initiated by contacting the breast based on a starting point on the curved surface and continuing by moving, or “rolling,” the curved surface over the surface of the breast to a final point. The rolling motion is characterized by placing the device at the starting point on the breast and, without sliding or skidding the device, moving to the final point on the breast in a consistent motion. For example, if the array of sensors has an arc length AB, the rolling motion will a substantially equal length AB of the breast surface. It should be appreciated by the person skilled in the art that the curved surface is configured to roll a variable linear distance, e.g., less than arc length AB, by rolling the curved surface from a starting point to a stopping point that is less than arc length AB, e.g., about 30%, about 40%, about 50%, about 60%, about 70%, about 80%, about 90%, or any percentage in between, of the arc length AB. In other words, the user is not required to roll the full arc length for every reading.

In some embodiments, the curved surface has a plurality of sizes, each having a different area of coverage. In some embodiments, the size of the curved surface size varies in arc length, in width, or both arc length and width. Accordingly, the number of rolling motions required to collect data over a fixed area of interest will vary. In some embodiments, in non-rolling use, the handheld device having a curved surface having a larger radius will have larger available area for non-rolling evaluation compared to the smaller radius. Accordingly, a smaller number of non-rolling presses cover a given area of breast using a handheld device having a curved surface having a larger radius. In some embodiments, in rolling use, the handheld device having a curved surface with a smaller radius has a sharper curvature resulting in higher local pressure on the lump. This will help in cases where the lump is only negligibly movable and requires more force to move. It should be appreciated by the person skilled in the art that ease of movement and maximum distance of movement may or may not be a criteria of interest, and instead the patient's pain threshold will determine if a smaller radius can be used to evaluate the parameters of movability. For example, if the lump is causing pain to the patient, a larger radius version would be more appropriate to use because it would cause less additional pain.

In some embodiments, the array of pressure sensors or electronic tactile sensors is a thin film sensor, e.g., a polymer-based thin film sensor. In some embodiments, the thin film sensor can be selected from a strain gauge-based semiconductor thin film sensor, a capacitive thin film sensor for detecting capacity change according to a pressure, a piezoresistive sensor using a piezo resistance effect, or other various pressure sensors. In some embodiments, the thin film sensor is based on the piezoresistive effect, and comprises a thin, flexible layer, e.g., a membrane or diaphragm, that changes in response to a force or deformation caused by an applied pressure. Changes in pressure result in a change in electrical resistance, which can be detected and converted into an electrical signal. Additional components in communication with the thin film sensor, e.g., contained in the housing, include, but are not limited to, data acquisition means, amplifiers, analog-to-digital converters, and signal conditioners. The data obtained from the thin film sensor can be relayed to software on a processing unit, either wirelessly using RF signals such as BLUETOOTH or other technologies or using a cable, e.g., USB or USB-C. The software is able to convert the electrical signals from the thin film sensor to pressure and display same in two-dimensions or three-dimensions on a display screen, as will be discussed further hereinbelow. It is understood that the person skilled in the art will understand how to select the appropriate thin film sensor for the handheld device and system described herein.

In some embodiments, the thin film sensor further comprises a thin, tactile coating to minimize or mitigate sliding over the skin of the breast.

In some embodiments, the handheld device and system comprising same is capable of detecting a lump in breast tissue that is less than about 4 mm in size, or less than about 3 mm in size, or less than about 2 mm in size, or less than about 1 mm in size.

In some embodiments, the handheld device and system comprising same are devoid of transducer elements. In some embodiments, the handheld device and system comprising same are devoid of force sensing arrays that emanate oscillatory signals. In some embodiments, the handheld device and system comprising same are devoid of electric piezoelectric finger sensors that comprise an electrode for providing a force to the finger. In some embodiments, the handheld device and system comprising same are devoid of a thin film comprising gold and/or CdS nanoparticles. In some embodiments, the handheld device and system comprising same does not comprise an array of pressure sensors or electronic tactile sensors positioned on a flat surface, e.g., a surface lacking a radius and an arc length. Instead, in some embodiments, the handheld device and system comprising same is capable of both rolling and non-rolling use for the collection of both rolling and non-rolling data.

Advantageously, the handheld device will have the capability to generate a detailed 3D spatiotemporal map of any detected lump and track its growth/movement longitudinally in subsequent screening tests. At least one major innovation associated with this technology is the use of the roller concept to observe the movability of the lump, providing insights and a probabilistic understanding of whether the lump is cancerous or non-cancerous. It is not possible to “roll” a handheld device having a flat surface comprising sensors, e.g., as disclosed in the prior art, and observe the moveability of the lump.

In a third aspect, a method for determining the presence, position, and movability of lumps in breast tissue is described, the method comprising using the handheld device of the first aspect or the system of the second aspect to collect rolling and non-rolling data and to analyze same to generate a report regarding risk of breast cancer.

In some embodiments, non-rolling data is collected by pressing the curved surface perpendicularly against a breast surface, such that the differences in pressure response between normal breast tissue and a lump, due to varying softness/hardness of the lump, are recorded to detect the probable presence of the lump. The non-rolling data collection is analogous to manual palpation performed by an examiner using finger pads to apply inward pressure on the lump. In some embodiments, the curved surface is pressed perpendicularly against a breast surface in at least 5, 10, 15, 20, 25 or more different locations on each breast, or any number therebetween, depending on the size of the breast and the area of the curved surface of the handheld device.

In some embodiments, the rolling data is collected subsequent to the probable detection of the lump (as determined using non-rolling data collection), wherein the curved surface is rolled over the region in which the probable lump was detected. In some embodiments, the rolling data is collected using the same handheld device or system comprising same that was used to collected the non-rolling data. During the rolling motion, pressure is applied to the probable lump in a lateral direction, as opposed to the inward pressure applied in the non-rolling method, thereby displacing the lump along the direction of the roll. The extent of displacement of the lump is measured to evaluate its movability. In some embodiments, the rolling motion is performed from at least two directions, the directions being defined along a vector line corresponding to a diameter of an imaginary circle centered at the location of the detected lump. Each vector line defines two rolling directions: from a first end to a second end and from the second end to the first end, thereby enabling assessment of lump characteristics from multiple orientations. In some embodiments, the rolling motion is performed on more than one vector line to provide a more accurate evaluation of the moveability of the lump. In some embodiments, two, three, four, five or more vector lines are evaluated by performing the rolling motion from at least two directions over the lump. It should be appreciated that, in general, the more moveable the lump, the increased likelihood that the lump is benign.

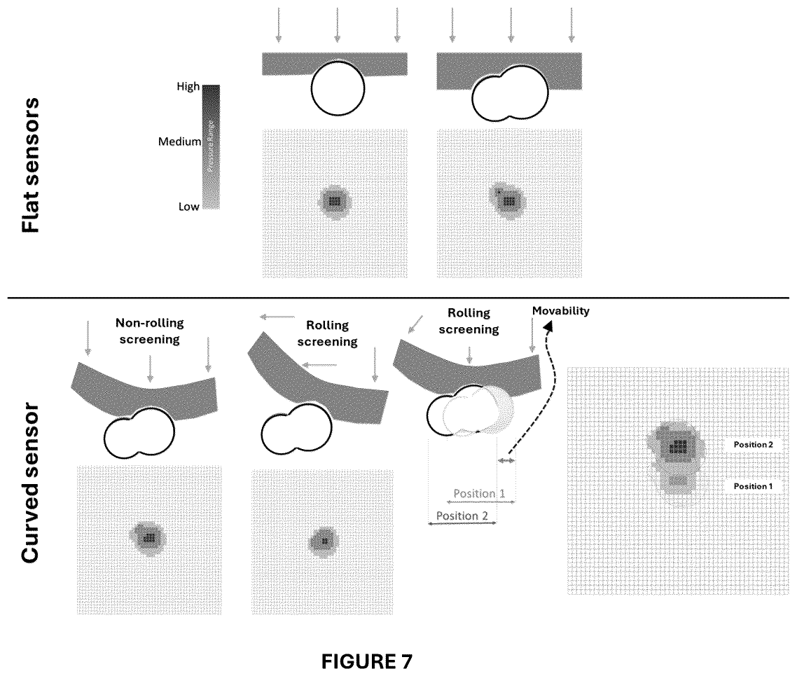

FIG. 7 is a schematic comparing the lump characterization capabilities of a flat sensor versus the curved rolling sensor described herein. The bottom sequence demonstrates the non-rolling screening with curved sensors used to screen for lumps and rolling screening used to determine the movability of the lump in one direction of rolling motion.

In some embodiments of the third aspect, a method for determining the presence, position, and movability of lumps in breast tissue is described, the method comprising:
using a handheld device to collect non-rolling data to obtain a 3D map of breast tissue to determine if a lump is present in the breast tissue; and
if a lump is detected, using the handheld device to collect rolling data in proximity of the lump to obtain a 3D map of movability of the lump to evaluate malignancy of the lump.

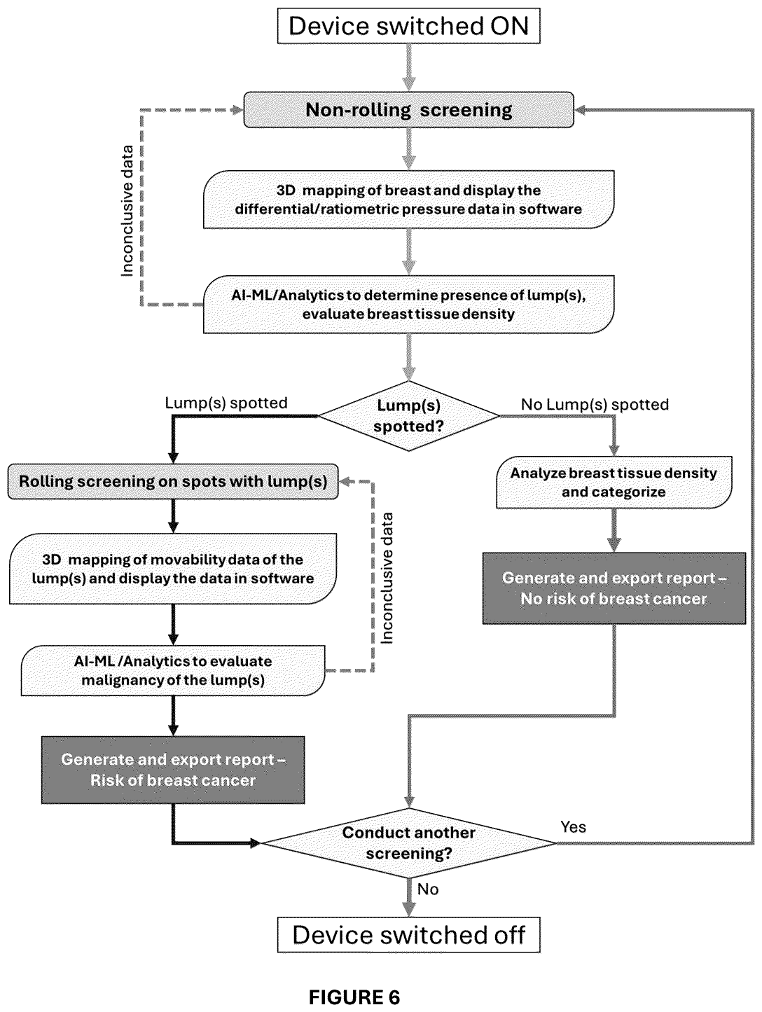

FIG. 6 is a flowchart detailing actions performed by the operator during the screening procedure, the data collection, and data analysis. As noted herein, in some embodiments, non-rolling data is collected first by pressing the curved surface of the handheld device perpendicularly against the breast at multiple locations. Using the system and method described herein, the non-rolling screening yields a 3D map of the differential/ratiometric pressure data of the breast tissue and using AI/ML, a lump, if present, is detected. In addition, breast tissue density can be evaluated. If no lump is detected, a report that no lumps have been detected can be generated and another screening can be performed, e.g., on the other breast. If a lump is detected, the user collects rolling data in proximity of the detected lump to evaluate the movability, and hence the possible malignancy, of the lump. Using the system and method described herein, the rolling screening yields a 3D map of the movability data of the lump and using AI/ML the malignancy of the lump is evaluated. A report that relating to the risk of breast cancer based on the movability/malignancy of the detected lump can be generated and another screening can be performed, e.g., on the other breast.

Advantageously, AI and ML offer the promise of faster results, greater precision, and the recognition of previously unappreciated complex correlations between variables with real-world impact.

In some embodiments, the system and method of using same creates a 3D model, which is a visual representation of any detected lump, wherein the handheld device of the system acquires new non-rolling medical imaging data; a computer processor is communicatively connected to the device and is configured to process the new non-rolling medical imaging data against an AI model, wherein said AI model is trained so that when it is deployed, the computer processor can identify a lump in the breast tissue, if present, from the non-rolling medical imaging data. If a lump is identified, using the AI model, a corresponding 3D model is selected and displayed on a display device configured to display to a system user at least the 3D model of the lump. Further, if a lump is detected, the system and method of using can acquire new rolling medical imaging data; a computer processor is communicatively connected to the device and is configured to process the new rolling medical imaging data against an AI model, wherein said AI model is trained so that when it is deployed, the computer processor can identify the movability and hence malignancy of the lump from rolling medical imaging data. Using the AI model, a corresponding 3D model is selected and displayed on a display device configured to display to a system user at least the 3D model of the evaluated malignancy.

In some embodiments, the AI model is trained using a dataset comprising a plurality of example images, including images of lumps in breast tissue and breast tissue devoid of lumps. The AI model can be trained to learn to classify patients as having a probable lump, or not, based on image data within the breast tissue, regardless of breast tissue density. Further, the AI model can be trained to classify the malignancy of any detected lump based on the movability of the lump within the breast tissue, regardless of breast tissue density. Additionally, in some embodiments, the AI model is trained to detect lumps and determine malignancy in augmented breasts, e.g., breasts comprising implants.

Accordingly in some embodiments of the third aspect, a method for determining the presence, position, and movability of lumps in breast tissue is described, the method comprising:
using a handheld device to collect non-rolling data to obtain a 3D map of breast tissue to determine if a lump is present in the breast tissue; and
if a lump is detected, using the handheld device to collect rolling data in proximity of the lump to obtain a 3D map of movability of the lump to evaluate malignancy of the lump,
wherein the handheld device comprises a housing comprising a curved surface comprising an array of pressure sensors or electronic tactile sensors.

In some other embodiments of the third aspect, a method for determining the presence and position of lumps in breast tissue is described, the method comprising:
using a handheld device to collect non-rolling data;
using the non-rolling data to obtain a 3D map of differential/ratiometric pressure data of the breast tissue to obtain a 3D map of breast tissue;
using AI/ML to detect the presence and position of a lump, if present, in the breast tissue.

In some other embodiments of the third aspect, a method for determining the presence and position of lumps in breast tissue is described, the method comprising:
using a handheld device to collect non-rolling data;
using the non-rolling data to obtain a 3D map of differential/ratiometric pressure data of the breast tissue to obtain a 3D map of breast tissue;
using AI/ML to detect the presence and position of a lump, if present, in the breast tissue,
wherein the handheld device comprises a housing comprising a curved surface comprising an array of pressure sensors or electronic tactile sensors.

In some other embodiments of the third aspect, a method for determining the presence, position, and movability of lumps in breast tissue is described, the method comprising:
using a handheld device to collect non-rolling data;
using the non-rolling data to obtain a 3D map of differential/ratiometric pressure data of the breast tissue to obtain a 3D map of breast tissue;
using AI/ML to detect the presence and position of a lump in the breast tissue;
using the handheld device to collect rolling data in proximity of the lump;
using the rolling data to obtain a 3D map of movability of the lump; and
using AI/ML to evaluate malignancy of the lump.

In some other embodiments of the third aspect, a method for determining the presence, position, and movability of lumps in breast tissue is described, the method comprising:
using a handheld device to collect non-rolling data;
using the non-rolling data to obtain a 3D map of differential/ratiometric pressure data of the breast tissue to obtain a 3D map of breast tissue;
using AI/ML to detect the presence and position of a lump in the breast tissue;
using the handheld device to collect rolling data in proximity of the lump;
using the rolling data to obtain a 3D map of movability of the lump; and
using AI/ML to evaluate malignancy of the lump,
wherein the handheld device comprises a housing comprising a curved surface comprising an array of pressure sensors or electronic tactile sensors.

Advantageously, the handheld device, system comprising same, and method of using same are configured to provide (i) the ability to detect the presence of one or more lumps, and if a lump is detected, (ii) localization of the lump, (iii) evaluation of the movability of the lump, and (iv) classification of breast density. In some embodiments, the movability of the lump, as determined by the device and system, is indicative of the potential malignancy of the lump. In some embodiments, the assessment of breast density is performed by subtracting the pressure values associated with normal breast tissue from the higher pressure values associated with the detected lumps, thereby enabling an overall classification of breast tissue density. The handheld device, system comprising same, and method of using same provided improved screening for breast cancer, providing for early diagnosis and intervention, and a reduced mortality risk. Further, the handheld device, system comprising same, and method of using same can be used in the comfort of a user's home, providing a high-quality breast screening experience.

Accordingly, in a fourth aspect, a method of detecting breast cancer in a subject is disclosed, said method comprising using the handheld device of the first aspect or the system of the second aspect to collect rolling and non-rolling data and to analyze same to generate a report regarding risk of breast cancer. In some embodiments, the more moveable the lump, the lower the risk of breast cancer.

In some embodiments, a method of detecting breast cancer in a subject is disclosed, said method comprising:
using a handheld device to collect non-rolling data to obtain a 3D map of breast tissue to determine if a lump is present in the breast tissue; and
if a lump is detected, using the handheld device to collect rolling data in proximity of the lump to obtain a 3D map of movability of the lump to evaluate malignancy of the lump.

In some embodiments, a method of detecting breast cancer in a subject is disclosed, said method comprising:
using a handheld device to collect non-rolling data to obtain a 3D map of breast tissue to determine if a lump is present in the breast tissue; and
if a lump is detected, using the handheld device to collect rolling data in proximity of the lump to obtain a 3D map of movability of the lump to evaluate malignancy of the lump,
wherein the handheld device comprises a housing comprising a curved surface comprising an array of pressure sensors or electronic tactile sensors.

In some other embodiments of the fourth aspect, a method of detecting breast cancer in a subject is described, the method comprising:
using a handheld device to collect non-rolling data;
using the non-rolling data to obtain a 3D map of differential/ratiometric pressure data of the breast tissue to obtain a 3D map of breast tissue;
using AI/ML to detect the presence and position of a lump, if present, in the breast tissue.

In some other embodiments of the fourth aspect, a method of detecting breast cancer in a subject is described, the method comprising:
using a handheld device to collect non-rolling data;
using the non-rolling data to obtain a 3D map of differential/ratiometric pressure data of the breast tissue to obtain a 3D map of breast tissue;
using AI/ML to detect the presence and position of a lump, if present, in the breast tissue,
wherein the handheld device comprises a housing comprising a curved surface comprising an array of pressure sensors or electronic tactile sensors.

In some other embodiments of the fourth aspect, a method of detecting breast cancer in a subject is described, the method comprising:
using a handheld device to collect non-rolling data;
using the non-rolling data to obtain a 3D map of differential/ratiometric pressure data of the breast tissue to obtain a 3D map of breast tissue;
using AI/ML to detect the presence and position of a lump in the breast tissue;
using the handheld device to collect rolling data in proximity of the lump;
using the rolling data to obtain a 3D map of movability of the lump; and
using AI/ML to evaluate malignancy of the lump.

In some other embodiments of the fourth aspect, a method of detecting breast cancer in a subject is described, the method comprising:
using a handheld device to collect non-rolling data;
using non-rolling data to obtain a 3D map of differential/ratiometric pressure data of the breast tissue to obtain a 3D map of breast tissue;
using AI/ML to detect the presence and position of a lump in the breast tissue;
using the handheld device to collect rolling data in proximity of the lump;
using the rolling data to obtain a 3D map of movability of the lump; and
using AI/ML to evaluate malignancy of the lump,
wherein the handheld device comprises a housing comprising a curved surface comprising an array of pressure sensors or electronic tactile sensors.

In some embodiments, the methods of the third and fourth embodiment are practiced without applying any electrical signals to the subject. In some embodiments, the methods of the third and fourth embodiment are practiced without any requirement to estimate the position of the probe/device relative to a known anatomical landmark on the examined subject. In some embodiments, the methods of the third and fourth embodiment are practiced without the application of any predetermined minimum pressure. In some embodiments, the methods of the third and fourth embodiment are practiced without the application of shear forces.

It should be appreciated by the person skilled in the art that an advantage of the methods described herein is that the non-skilled medical user of the handheld device can perform periodic scans of their breast tissue by collecting non-rolling and when relevant, rolling data, and if a lump is detected, seek a professional medical opinion. The methods described herein provide a less subjective detection of lumps, as well as the movability of same, relative to the digits of a human hand, and permit earlier diagnosis of breast cancer and earlier treatment of same.

### Computer Program Products

The present subject matter described herein may be a method and/or a computer program product. In some embodiments, the computer program product may include a computer readable storage medium (or media) having computer readable program instructions thereon for causing a processor to carry out aspects of the present subject matter.

In some embodiments, the computer readable storage medium can be a tangible device that can retain and store instructions for use by an instruction execution device. The computer readable storage medium may be, for example, but is not limited to, an electronic storage device, a magnetic storage device, an optical storage device, an electromagnetic storage device, a semiconductor storage device, or any suitable combination of the foregoing. A non-exhaustive list of more specific examples of the computer readable storage medium includes the following: a portable computer diskette, a hard disk, a RAM, a ROM, an erasable programmable read-only memory (EPROM or Flash memory), a static random access memory (SRAM), a portable compact disc read-only memory (CD-ROM), a digital versatile disk (DVD), a memory stick, a floppy disk, a mechanically encoded device such as punch-cards or raised structures in a groove having instructions recorded thereon, and any suitable combination of the foregoing. A computer readable storage medium, as used herein, is not to be construed as being transitory signals per se, such as radio waves or other freely propagating electromagnetic waves, electromagnetic waves propagating through a waveguide or other transmission media (e.g., light pulses passing through a fiber-optic cable), or electrical signals transmitted through a wire.

In some embodiments, computer readable program instructions described herein can be downloaded to respective computing/processing devices from a computer readable storage medium or to an external computer or external storage device via a network, for example, the Internet, a local area network, a wide area network and/or a wireless network, or Near Field Communication. The network may comprise copper transmission cables, optical transmission fibers, wireless transmission, routers, firewalls, switches, gateway computers and/or edge servers. A network adapter card or network interface in each computing/processing device receives computer readable program instructions from the network and forwards the computer readable program instructions for storage in a computer readable storage medium within the respective computing/processing device.

In some embodiments, computer readable program instructions for carrying out operations of the present subject matter may be assembler instructions, instruction-set-architecture (ISA) instructions, machine instructions, machine dependent instructions, microcode, firmware instructions, state-setting data, or either source code or object code written in any combination of one or more programming languages, including an object oriented programming language such as Java, Smalltalk, C++, Javascript or the like, and conventional procedural programming languages, such as the “C” programming language or similar programming languages. The computer readable program instructions may execute entirely on the user's computer, partly on the user's computer, as a stand-alone software package, partly on the user's computer and partly on a remote computer or entirely on the remote computer or server. In the latter scenario, the remote computer may be connected to the user's computer through any type of network, including a local area network (LAN) or a wide area network (WAN), or the connection may be made to an external computer (for example, through the Internet using an Internet Service Provider). In some embodiments, electronic circuitry including, for example, programmable logic circuitry, field-programmable gate arrays (FPGA), or programmable logic arrays (PLA) may execute the computer readable program instructions by utilizing state information of the computer readable program instructions to personalize the electronic circuitry, in order to perform aspects of the present subject matter.

In some embodiments, the computer readable program instructions may be provided to a processor of a computer, special purpose computer, or other programmable data processing apparatus to produce a machine, such that the instructions, which execute via the processor of the computer or other programmable data processing apparatus, create means for implementing the functions/acts specified in the flowchart and/or block diagram block or blocks. In some embodiments, the computer readable program instructions may also be stored in a computer readable storage medium that can direct a computer, a programmable data processing apparatus, and/or other devices to function in a particular manner, such that the computer readable storage medium having instructions stored therein comprises an article of manufacture including instructions which implement aspects of the function/act specified in the flowchart and/or block diagram block or blocks.

In some embodiments, the computer readable program instructions may also be loaded onto a computer, other programmable data processing apparatus, or other device to cause a series of operational steps to be performed on the computer, other programmable apparatus or other device to produce a computer implemented process, such that the instructions which execute on the computer, other programmable apparatus, or other device implement the functions/acts specified in the flowchart and/or block diagram block or blocks.

Certain embodiments described herein make use of (e.g., include) software instructions that include one or more machine learning module(s), also referred to herein as artificial intelligence software. As used herein, the term “machine learning module” refers to a computer implemented process (e.g., function) that implements one or more specific machine learning algorithms in order to determine, for a given input (such as an image (e.g., a 2D image; e.g., a 3D image), dataset, and the like) one or more output values. For example, a machine learning module may receive as input a 3D image of an object, and for each voxel of the image, determine a value that represents a likelihood of a lump in the breast tissue of the subject. In certain embodiments, two or more machine learning modules may be combined and implemented as a single module and/or a single software application. In certain embodiments, two or more machine learning modules may also be implemented separately, e.g., as separate software applications. A machine learning module may be software and/or hardware. For example, a machine learning module may be implemented entirely as software, or certain functions may be carried out via specialized hardware (e.g., via an application specific integrated circuit (ASIC)).

Accordingly, in a fourth aspect, a computer-readable media storing computer-readable instructions is described, which, when executed by a processor, cause the processor to process new non-rolling medical imaging data against an AI model, wherein said AI model is trained so that when it is deployed, the computer processor can identify a lump in breast tissue, if present, from the new non-rolling medical imaging data. Further, computer-readable media storing computer-readable instructions executed by a processor can cause the processor to select a 3D model and display same. Further, a computer-readable media storing computer-readable instructions is described, which, when executed by a processor, cause the processor to process new rolling medical imaging data against an AI model, wherein said AI model is trained so that when it is deployed, the computer processor can identify the movability and malignancy of the lump in the breast tissue from the new rolling medical imaging data.

### CLAUSES

Clause 1: A system for detecting a lump in breast tissue, said system comprising:
a handheld device comprising a housing comprising a curved surface comprising an array of pressure sensors or electronic tactile sensors;
a processor configured to control the sensors and collect data therefrom; and
software configured to display and analyze the compiled data.

Clause 2: The system of clause 1, wherein a curvature of the curved surface is defined by an arc length and a radius.

Clause 3: The system of clause 2, wherein the arc length is defined by the outermost sensors along the curved surface in the array.

Clause 4: The system of clause 1, wherein the array of pressure sensors or electronic tactile sensors is a thin film sensor.

Clause 5: The system of clause 4, wherein the thin film sensor is based on the piezoresistive effect, and comprises a thin, flexible layer that changes in response to a force or deformation caused by an applied pressure.

Clause 6: The system of clause 1, wherein the system generates a three-dimensional (3D) spatiotemporal map of a detected lump and tracks its growth and longitudinal movement over time.

Clause 7: A method for determining a presence, position, and movability of a lump in breast tissue, the method comprising:
using the system of clause 1 to collect non-rolling data to obtain a 3D map of breast tissue to determine if a lump is present in the breast tissue; and
if a lump is detected, using the system to collect rolling data in proximity of the lump to obtain a 3D map of movability of the lump to evaluate malignancy of the lump.

Clause 8: The method of clause 7, wherein the non-rolling data is collected by pressing the curved surface perpendicularly against a breast surface, such that the differences in pressure response between normal breast tissue and a lump are recorded to detect the presence of the lump.

Clause 9: The method of clause 7, wherein the rolling data is collected subsequent to the detection of the lump, wherein the curved surface is rolled over the region in which the probable lump was detected.

Clause 10: The method of clause 9, wherein pressure is applied to the lump in a lateral direction, thereby displacing the lump along the direction of the roll and the extent of displacement of the lump is used to evaluate lump movability.

Clause 11: The method of clause 10, wherein a rolling motion is performed from at least two directions, the directions being defined along a vector line corresponding to a diameter of an imaginary circle centered at the location of the detected lump.

Clause 12: The method of clause 11, wherein the rolling motion is performed without sliding or skidding the handheld device over the breast tissue.

Clause 13: The method of clause 7, wherein the rolling data is collected using the same handheld device that was used to collect the non-rolling data.

Clause 14: The method of clause 7, wherein the system is capable of detecting a lump in breast tissue that is less than about 4 mm in size.

Clause 15: The method of clause 7, wherein artificial intelligence (AI)/machine learning (ML) is used to detect the presence and position of a lump, if present, in the breast tissue.

Clause 16: The method of clause 7, wherein AI/ML is used to evaluate malignancy of the lump.

Clause 17: A method of detecting breast cancer in a subject, the method comprising:
using the system of clause 1 to collect non-rolling data;
using non-rolling data to obtain a 3D map of differential/ratiometric pressure data of the breast tissue to obtain a 3D map of breast tissue;
using AI/ML to detect the presence and position of a lump in the breast tissue;
using the handheld device to collect rolling data in proximity of the lump;
using the rolling data to obtain a 3D map of movability of the lump; and
using AI/ML to evaluate malignancy of the lump.

Although the invention has been variously disclosed herein with reference to illustrative embodiments and features, it will be appreciated that the embodiments and features described hereinabove are not intended to limit the invention, and that other variations, modifications and other embodiments will suggest themselves to those of ordinary skill in the art, based on the disclosure herein. The invention therefore is to be broadly construed, as encompassing all such variations, modifications and alternative embodiments within the spirit and scope of the claims hereafter set forth.

What is claimed is:

1. A system for detecting a lump in breast tissue, said system comprising:
a handheld device comprising a housing comprising a curved surface comprising an array of pressure sensors or electronic tactile sensors;
a processor configured to control the sensors and collect data therefrom; and
software configured to display and analyze the compiled data.

2. The system of claim 1, wherein a curvature of the curved surface is defined by an arc length and a radius.

3. The system of claim 2, wherein the arc length is defined by the outermost sensors along the curved surface in the array.

4. The system of claim 1, wherein the array of pressure sensors or electronic tactile sensors is a thin film sensor.

5. The system of claim 4, wherein the thin film sensor is based on the piezoresistive effect, and comprises a thin, flexible layer that changes in response to a force or deformation caused by an applied pressure.

6. The system of claim 1, wherein the system generates a three-dimensional (3D) spatiotemporal map of a detected lump and tracks its growth and longitudinal movement over time.

7. A method for determining a presence, position, and movability of a lump in breast tissue, the method comprising:
using the system of claim 1 to collect non-rolling data to obtain a 3D map of breast tissue to determine if a lump is present in the breast tissue; and
if a lump is detected, using the system to collect rolling data in proximity of the lump to obtain a 3D map of movability of the lump to evaluate malignancy of the lump.

8. The method of claim 7, wherein the non-rolling data is collected by pressing the curved surface perpendicularly against a breast surface, such that the differences in pressure response between normal breast tissue and a lump are recorded to detect the presence of the lump.

9. The method of claim 7, wherein the rolling data is collected subsequent to the detection of the lump, wherein the curved surface is rolled over the region in which the probable lump was detected.

10. The method of claim 9, wherein pressure is applied to the lump in a lateral direction, thereby displacing the lump along the direction of the roll and the extent of displacement of the lump is used to evaluate lump movability.

11. The method of claim 10, wherein a rolling motion is performed from at least two directions, the directions being defined along a vector line corresponding to a diameter of an imaginary circle centered at the location of the detected lump.

12. The method of claim 11, wherein the rolling motion is performed without sliding or skidding the handheld device over the breast tissue.

13. The method of claim 7, wherein the rolling data is collected using the same handheld device that was used to collect the non-rolling data.

14. The method of claim 7, wherein the system is capable of detecting a lump in breast tissue that is less than about 4 mm in size.

15. The method of claim 7, wherein artificial intelligence (AI)/machine learning (ML) is used to detect the presence and position of a lump, if present, in the breast tissue.

16. The method of claim 7, wherein AI/ML is used to evaluate malignancy of the lump.

17. A method of detecting breast cancer in a subject, the method comprising:
using the system of claim 1 to collect non-rolling data;
using non-rolling data to obtain a 3D map of differential/ratiometric pressure data of the breast tissue to obtain a 3D map of breast tissue;
using AI/ML to detect the presence and position of a lump in the breast tissue;
using the system to collect rolling data in proximity of the lump;
using the rolling data to obtain a 3D map of movability of the lump; and
using AI/ML to evaluate malignancy of the lump.
